(19) United States
(12) Patent Application Publication
(10) Pub. No.: US 2007/0237843 A1
(43) Pub. Date: Oct. 11, 2007

(54) Compositions for herbal teas enriched with dried plant extracts

(75) Inventor: Valentino Marcati, Sansepolcro (IT)

Correspondence Address: YOUNG & THOMPSON, 745 SOUTH 23RD STREET, 2ND FLOOR, ARLINGTON, VA, 22202, US

(73) Assignee: ABOCA S.P.A., Sansepolcro (IT)

(21) Appl. No.: 11/806,557

(22) Filed: Jun. 1, 2007

Related U.S. Application Data
(62) Division of application No. 11/100,464, filed on Apr. 7, 2005, now abandoned.

(30) Foreign Application Priority Data
Apr. 8, 2004 (IT) MI2004A000696

(51) Int. Cl. A61K 36/45; A61K 36/28; A61K 36/534; A61K 36/537; A61K 36/73; A61K 36/752; A61K 36/82

(52) U.S. Cl. 424/729; 424/732; 424/746; 424/747; 424/764; 424/765

(57) ABSTRACT

Disclosed are compositions for herbal teas including at least one plant material for conventional herbal teas and at least one extract in the form of a water-dispersible granulate obtainable by extraction from the material or a different plant material.

The present invention relates to herbal teas comprising at least one plant material for conventional herbal teas and at least one extract in the form of a water-dispersible granulate obtainable by extraction from said plant material or a different plant material. Said herbal teas can also be packaged ready for use in monodose filter bags.

The use of plant material to infuse in hot drinks for the preparation of tea and herbal teas has obviously been known for a very long time. With the introduction of filter bags onto the market, the use of herbal teas has increased in view of their practicality. In general, said bags consist of a porous, permeable fabric, usually made of a cellulose material, into which appropriate quantities of plant material suitable for the preparation of herbal teas are inserted. Examples of plant materials commonly used in herbal teas are green or black tea leaves, camomile flowers, butcher's broom and black cohosh roots, passion flower tips, dog rose fruits, karkadé calyxes, mallow leaves and flowers, lime flowers and floral bracts, mint leaves, lemon balm leaves, verbena leaves, fennel seeds, etc.

Said plant materials are dried and chopped before use to form what is known as the “herbal tea cut”.

Herbal teas are a particularly pleasant form of administration of phytotherapeutic active ingredients; however, various problems are involved which limit their application possibilities.

For example, a traditional herbal tea is hard to standardize in terms of active ingredients, due to the natural variability of the plants that make it up and to the different time and temperature conditions of the infusion made by the user. Among other things, active ingredients with a mainly lipophilic nature are only extracted to a minimal extent by the hot water normally used to make herbal teas.

Not all the plants used for their activity in the formula of herbal teas have a pleasant flavour, so it is often necessary to include in the composition a plant or mixture of plants which make the flavour of the product more acceptable.

These problems are solved according to this invention by adding to plant material for traditional herbal teas suitable amounts of dried extracts of the same or other plants, in order to:
enrich it with active ingredients until it contains quantities similar to those obtainable with other concentrated forms of administration such as tablets, capsules, drops and fluids
provide a standard dose of the active ingredients characterizing the desired activity profile
allow the administration in the form of herbal teas even of active ingredients which cannot be extracted under normal herbal tea preparation conditions due to their mainly lipophilic characteristics
improve the organoleptic characteristics of the product.

The extracts are preferably freeze-dried aqueous or hydroethanolic extracts, and are used to produce granulates which can be obtained with known techniques, as described, for example, in EP 1075830.

According to said granulation technique, the dried extracts and/or powders of one or more medicinal herbs are first mixed in a suitable ratio. Said mixture is made to fall regularly from a hopper, and a jet of steam at atmospheric pressure is directed at it for a few fractions of a second, followed by the introduction of hot air. In this way the steam is eliminated and a drying process that consolidates the granules produced is happened. The process described produces a product without the unfavourable hygroscopic characteristics of the starting extracts and with excellent flowability, and does not require the use of technological adjuvants to stabilize the granules, which consequently consist of 100% plant ingredients, the mixtures of which are specifically formulated according to the required use.

The particle size of the granulates obtained is compatible with their use mixed with the various “herbal tea cuts” of traditional plants.

The herbal teas and filter bags do not require any adaptation, and can be packaged with conventional plant and machinery.

Examples of plant materials for conventional herbal teas include black cohosh, passion flower, sage, liquorice, star anise, butcher's broom, camomile, centella, witch hazel, red vine, mint, green tea, black tea, fennel, lime, lemon balm, mallow, dog rose, ginkgo, karkadé, senna, java tea, rooibos tea, bilberry, goldenrod, grindelia, artichoke and dandelion.

Examples of dried extracts that can be added to conventional materials which are dried and/or chopped but not subjected to extraction treatments are extracts of black cohosh, passion flower, butcher's broom, red vine, camomile, ginkgo, senna, dandelion, willow, meadowsweet, milk thistle, echinacea, ginseng, sage, dropwort and everlasting flower.

Examples of granulates containing the dried extracts in a mixture are black cohosh root dry extract 50%+passion flower dry extract 50%; butcher's broom root dry extract 21.8%+red vine leaves dry extract 31.3%+powdered peppermint 46.9%; dandelion root dry extract 68%+milk thistle seeds dry extract 10%+java tea leaves dry extract 22%; camomile flowers dry extract 80%+powdered lemon balm leaves 20%.

The herbal teas according to the invention are packaged in 1 kg packs, suitably protected against light and humidity; the extracts can be present in percentages ranging between approx. 2% and approx. 30% in weight of the total content. The recommended dose is 1 to 5 g of composition in 150 ml of water.

The sachets according to the invention generally take. the form of a monodose bag made of cotton or other cellulose material, and typically contain 0.5 to 4 g of the composition. The extracts can be present in percentages ranging between approx. 2% and approx. 30% in weight of the total content of the filter, obviously depending on the type of extract and the active ingredient content.

The following examples illustrate the invention in greater detail.

### COMPARATIVE EXAMPLE 1

Disclosed is a herbal tea characterized by the presence of black cohosh root as the main ingredient responsible for the activity of the product.

2 g of root with an 0.32% actein+deoxyactein content is placed in 150 ml of water at 90° C. for 5 minutes and filtered through a narrow-mesh strainer. The results of the concentrations of actein and deoxyactein extracted in the herbal tea compared with the theoretical value are set out in Table 1 below.

TABLE 1

| | Active | Theoretical | % of active |
|---|---|---|---|
| | ingredients | active | ingredients |
| Plant | found | ingredients | extracted |
| Black | Actein + deoxyactein | Actein + deoxyactein | 10% |
| cohosh | 0.004 mg/ml | 0.043 mg/ml | |

### COMPARATIVE EXAMPLE 2

Herbal Tea

Composition per 100 g of product:

| INGREDIENTS | % |
|---|---|
| Black cohosh herbal tea cut | 31.25 |
| Passion flower herbal tea cut | 31 |
| Liquorice herbal tea cut | 27 |
| Sage herbal tea cut | 8 |
| Star anise herbal tea cut | 2.75 |

2 g of herbal tea is placed in 150 ml of water at 90° C. for 5 minutes, and filtered through a narrow-mesh strainer. The results of the concentrations of actein and deoxyactein extracted in the composite herbal tea compared with the theoretical value are set out in Table 2 below.

TABLE 2

| | Active | Theoretical | % of active |
|---|---|---|---|
| | ingredients | active | ingredients |
| Plant | found | ingredients | extracted |
| Black | Actein + deoxyactein | Actein + deoxyactein | 11.5% |
| cohosh | 0.0015 mg/ml | 0.013 mg/ml | |

### EXAMPLE 1

Herbal Tea

Composition per 100 g of product:

| INGREDIENTS | % |
|---|---|
| Passion flower herbal tea cut | 31 |
| Liquorice herbal tea cut | 27 |
| Star anise herbal tea cut | 25 |
| Granulate constituted by black cohosh root dry extract 50% | 9 |
| (actein + deoxyactein content: 2.23%) + passion flower | |
| dry extract 50% | |
| Sage herbal tea cut | 8 |

2 g of herbal tea is placed in 150 ml of water at 90° C. for 5 minutes, and filtered through a narrow-mesh strainer. The results of the concentrations of actein and deoxyactein extracted in the composite herbal tea compared with the theoretical value are set out in Table 3 below.

TABLE 3

| | Active | Theoretical | % of active |
|---|---|---|---|
| | ingredients | active | ingredients |
| Plant | found | ingredients | extracted |
| Black | Actein + deoxyactein | Actein + deoxyactein | 91.5% |
| cohosh | 0.0119 mg/ml | 0.013 mg/ml | |

The data reported above demonstrate that the filter bags containing the extracts according to the invention guarantee the standardization of the product, and above all allow much greater extraction of the active ingredients.

### COMPARATIVE EXAMPLE 3

This example describes herbal teas based on butcher's broom and red vine. A filter containing 2 g of butcher's broom root with a 2.5% saponins content is immersed in 150 ml of water at 90° C. for 5 minutes. The results of the concentrations of saponins extracted in the herbal tea compared with the theoretical value are set out in Table 4 below.

TABLE 4

| | Active | Theoretical | % of active |
|---|---|---|---|
| | ingredients | active | ingredients |
| Plant | found | ingredients | extracted |
| Butcher's | Total saponins | Total saponins | 56% |
| broom | 1.86 mg/ml | 3.33 mg/ml | |

A filter containing 2 g of red vine leaves with an 0.4% anthocyanins content is immersed in 150 ml of water at 90° C. for 5 minutes. The results of the anthocyanins concentrations extracted in the herbal tea compared with the theoretical value are set out in Table 5 below.

TABLE 5

| | Active | Theoretical | % of active |
|---|---|---|---|
| | ingredients | active | ingredients |
| Plant | found | ingredients | extracted |
| Red vine | Total anthocyanins | Total anthocyanins | 44% |
| | 0.023 mg/ml | 0.053 mg/ml | |

### COMPARATIVE EXAMPLE 4

A 2 g filter bag has the following percentage composition:

| INGREDIENTS | % |
|---|---|
| Butcher's broom herbal tea cut | 34 |
| Liquorice herbal tea cut | 18 |
| Red vine herbal tea cut | 17 |
| Centella herbal tea cut | 16 |
| Witch hazel herbal tea cut | 15 |

The 2 g filter is immersed in 150 ml of water at 90° C. for 5 minutes. The results of the saponins and anthocyanins concentrations extracted in the herbal tea compared with the theoretical value are set out in Table 6 below.

TABLE 6

| | Active | Theoretical | % of active |
|---|---|---|---|
| | ingredients | active | ingredients |
| Plant | found | ingredients | extracted |
| Butcher's | Total saponins | Total saponins | 50.4% |
| broom | 0.057 mg/ml | 0.113 mg/ml | |
| Red vine | Total anthocyanins | Total anthocyanins | 38% |
| | 0.0038 mg/ml | 0.01 mg/ml | |

### EXAMPLE 2

2 g Filter Sachet

Composition per 100 g of product:

| INGREDIENTS | % |
|---|---|
| Butcher's broom herbal tea cut | 20 |
| Liquorice herbal tea cut | 18.5 |
| Granulate constituted by butcher's broom dry extract | 16 |
| (saponins content: 10%) 21.8% + red vine dry extract | |
| (anthocyanins content: 1.3%) 31.3% + powdered | |
| peppermint 46.9% | |
| Centella herbal tea cut | 16 |
| Witch hazel herbal tea cut | 15 |
| Red vine herbal tea cut | 14.5 |

The filter is immersed in 150 ml of water at 90° C. for 5 minutes. The results of the saponins and anthocyanins concentrations extracted in the herbal tea compared with the theoretical value are set out in Table 7 below.

TABLE 7

| | Active | Theoretical | % of active |
|---|---|---|---|
| | ingredients | active | ingredients |
| Plant | found | ingredients | extracted |
| Butcher's | Total saponins | Total saponins | 98.2% |
| broom | 0.111 mg/ml | 0.113 mg/ml | |
| Red vine | Total anthocyanins | Total anthocyanins | 96% |
| | 0.0096 mg/ml | 0.01 mg/ml | |

The data reported above demonstrate that the filter bags containing the extracts according to the invention guarantee the standardization of the product, and above all allow much greater extraction of the active ingredients.

A further advantage is that the desired quantity of plants useful to improve the flavour of the herbal tea (such as peppermint) can be added.

1. An herbal tea, comprising:
i) at least one herbal plant material dried and cut in herbal tea cut form, and
ii) at least one extract in the form of a water-dispersible granule, said extract comprising a standard dosage and concentrated amount of an active ingredient from said plant material or a different plant material.

2. A filter bag comprising the herbal tea according to claim 1.

3. The filter bag according to claim 2, wherein the bag is in the form of a single-dose cotton bag.

4. The herbal tea according to claim 17, wherein the extract is a freeze-dried aqueous or hydroethanolic extract.

5. The herbal tea according to claim 1, wherein the herbal plant material is selected from the group consisting of black cohosh, passion flower, sage, liquorice, star anise, butcher's broom, camomile, centella, witch hazel, red vine, mint, green tea, black tea, fennel, lime, lemon balm, mallow, dog rose, ginkgo, karkadé, senna, java tea, rooibos tea, bilberry, goldenrod, grindelia, artichoke and dandelion.

6. The herbal tea according to claim 1, wherein the extract is selected from the group consisting of black cohosh, passion flower, butcher's broom, red vine, camomile, ginkgo, senna, dandelion, willow, meadowsweet, milk thistle, echinacea, ginseng, sage, dropwort and everlasting flower.

7. The herbal tea according to claim 6, comprising black cohosh extract.

8. The herbal tea according to claim 6, comprising passion flower extract.

9. The herbal tea according to claim 6, comprising butcher's broom extract.

10. The herbal tea according to claim 6, comprising red vine extract.

11. The herbal tea according to claim 6, comprising camomile extract.

12. The herbal tea according to claim 6, comprising ginkgo extract.

13. The herbal tea according to claim 1, wherein the herbal plant material comprises black cohosh, passion flower, liquorice, sage and star anise, and the extract comprises black cohosh with concentrated amounts of actein and deoxyactein.

14. The herbal tea according to claim 1, wherein the herbal plant material is selected from the group consisting of black cohosh, passion flower, sage, liquorice, star anise, butcher's broom, camomile, centella, witch hazel, red vine, mint, green tea, black tea, fennel, lime, lemon balm, mallow, dog rose, ginkgo, karkadé, senna, java tea, rooibos tea, bilberry, goldenrod, grindelia, artichoke and dandelion; and the extract is selected from the group consisting of black cohosh, passion flower, butcher's broom, red vine, camomile, ginkgo, senna, dandelion, willow, meadowsweet, milk thistle, echinacea, ginseng, sage, dropwort and everlasting flower.

15. The herbal tea according to claim 1, wherein said extract is in an amount ranging between approximately 2% and 30% by weight of the total herbal tea.

16. A single-dose tea bag comprising the herbal tea according to claim 1 in an amount of about 0.5 to 4 g.

17. An herbal tea, comprising:
i) at least one herbal plant material dried and cut in herbal tea cut form, and
ii) at least one freeze-dried aqueous or hydroethanolic extract in the form of a water-dispersible granule, said extract comprising a standard dosage and concentrated amount of an active ingredient from said plant material or a different plant material, and wherein said extract is in an amount ranging between approximately 2% and 30% by weight of the total herbal tea.

18. A single-dose tea bag comprising the herbal tea according to claim 15 in an amount of about 0.5 to 4 g.

19. The herbal tea according to claim 15, wherein the herbal plant material is selected from the group consisting of black cohosh, passion flower, sage, liquorice, star anise, butcher's broom, camomile, centella, witch hazel, red vine, mint, green tea, black tea, fennel, lime, lemon balm, mallow, dog rose, ginkgo, karkadé, senna, java tea, rooibos tea, bilberry, goldenrod, grindelia, artichoke and dandelion.

20. The herbal tea according to claim 15, wherein the extract is selected from the group consisting of black cohosh, passion flower, butcher's broom, red vine, camomile, ginkgo, senna, dandelion, willow, meadowsweet, milk thistle, echinacea, ginseng, sage, dropwort and everlasting flower.
